(19) United States
(12) Patent Application Publication
(10) Pub. No.: US 2026/0158095 A1
(43) Pub. Date: Jun. 11, 2026

(54) PLANT EXTRACT AND USE THEREOF IN TOPICAL COMPOSITIONS

(71) Applicant: TERM HELVET SA, Zug (CH)

(72) Inventor: Jean-Hilaire Saurat, Geneva (CH)

(73) Assignee: TERM HELVET SA, Zug (CH)

(21) Appl. No.: 18/707,265

(22) PCT Filed: Nov. 2, 2022
(86) PCT No.: PCT/EP2022/080600

§ 371 Date: May 3, 2024

(30) Foreign Application Priority Data
Nov. 3, 2021 (EP) 21206259.0

(51) Int. Cl. A61K 36/68; A61K 9/00; A61K 31/405; A61P 17/16

(52) U.S. Cl. CPC A61K 36/68; A61K 9/0014; A61K 31/405; A61P 17/16; A61K 2236/15; A61K 2236/333; A61K 2236/51

(57) ABSTRACT

The extract of plants of the Picrorhiza genus, chosen from Picrorhiza kurroa, Picrorhiza scrophulariiflora and Neopicrorhiza scrophulariiflora, and mixtures thereof, is obtained by extraction, with an organic solvent, of an acidic aqueous hydrolysate of elements of the underground part of the plants, the elements being chosen from the rhizomes and the roots. The extract has cosmetic or dermatological use, in a topical formulation, for treating normal or pathological ageing of the skin.

### FIELD OF THE INVENTION

The invention relates to a novel plant extract and to its use in topical compositions.

The invention has applications in the cosmetics field in the form of compositions for topical use, intended to counteract the visible signs of skin aging.

The invention has applications in the dermatological field in the form of compositions for topical use, intended to treat manifestations of the early stages of dermatoporosis.

### BACKGROUND ART

Skin aging is one of the most visible signs of the normal process of human involution. Topical cosmetic compositions intended to prevent premature skin aging, whether photoinduced or not, are designed to counteract thinning of skin tissue, reduce wrinkles, promote epidermal and dermal cell activity, and firm the skin by improving its elasticity and/or its state of hydration.

By way of example, mention may be made of compositions incorporating retinoids such as retinal, vitamins C or E, or Centella asiatica extract containing triterpene saponins, asiaticoside and madecassoside.

Despite the existence of numerous cosmetic preparations on the market, the need remains to develop other topical compositions intended to counteract the manifestations of skin aging, either by acting on a specific aging factor or on a more global syndrome.

However, in combination with advanced age, and also with other factors such as the use of corticosteroid-based treatments, a syndrome of functional and chronic cutaneous insufficiency may be seen, which goes far beyond the appearance problems mentioned above. J-H. Saurat proposed the holistic term “Dermatoporosis”, in documents [1] and [2], to cover all manifestations of this syndrome.

Summarizing the evolution of this condition, and according to the publications dealing with this condition, four stages are usually distinguished:
Stage 1: severe thinning of the skin with the appearance of senile purpura, followed by stellate pseudoscars,
Stage 2: small skin lacerations,
Stage 3: increase in skin lacerations,
Stage 4: appearance of dissecting hematomas.

Documents [1] to [8] cited in the attached bibliography deal with this subject in detail.

To counteract the first manifestations of stage 1, the use of hyaluronate has been proposed. Hyaluronate is synthesized in normal skin by dermal fibroblasts and epidermal keratinocytes. It promotes water retention in the skin's connective tissue and helps maintain skin elasticity. Its synthesis reduces with age. Topical administration of formulations based on hyaluronate fragments that penetrate the skin barrier allows an increase in the amount of hyaluronate present in situ, contributing toward maintaining the skin's state of hydration and thus firming it.

Document [9] proposes treating skin tissue atrophy with topical preparations combining specific hyaluronate fragments and a retinoid. However, said fragments may be difficult to prepare in their ideal molecular weight and said retinoids are irritant.

Document [10] teaches the use of AhR receptor agonist ligands in cutaneous tissues for the treatment of various skin disorders. In particular, said document proposes the use of L-tryptophan or certain short-lived derivatives thereof, i.e. metabolizable by phase I enzymes, to treat manifestations of dermatoporosis. The contents of said document are incorporated here by this reference [10].

### SUMMARY OF THE INVENTION

Despite the existence of numerous preparations on the market, the need remains to develop other compositions for topical use intended for preventing or treating this pathological aging of the skin, whether intrinsic or induced by corticosteroids, by acting on a specific aging factor, or on a more global syndrome.

The scientific name Picrorhiza kurroa Royle denotes a plant of the taxonomic genus Picrorhiza, which belongs to the Scrofulariaceae family. This plant grows in the Himalayas from Kashmir to Sikkim, at altitudes of between 2700 and 4500 meters. It is one of the oldest medicinal plants in Ayurvedic medicine, known by the local vernacular names kutki or katuki. The underground part of the plant, i.e. its rhizome and roots, is traditionally used in Ayurvedic medicine in oral administration to treat various digestive problems (reference [11]).

Since the essential components of the plant's rhizome and roots are on the whole sparingly soluble in water, and some of them extremely bitter, the underground part of the plant is usually used in dried form, ground to a powder and encapsulated for oral administration in gel capsules, rather than as an infusion. It has also been proposed for administration as a powder mixed with honey.

Uses are also mentioned for treating scorpion stings, eczema and vitiligo, likewise via oral administration.

The aim of the invention is to propose compositions for topical use, intended for treating, in humans, manifestations of skin aging as described above.

One subject of the invention is a novel extract of a plant of the Picrorhiza genus, namely an extract obtained by extraction with an organic solvent from an acidic aqueous hydrolyzate of elements from the underground part of said plant, chosen from its rhizome and roots.

Plants of the Picrorhiza genus, envisaged in the context of the invention, are in particular chosen from Picrorhiza kurroa, Picrorhiza scrophulariiflora and Neopicrorhiza scrophulariiflora and mixtures thereof.

In particular, the invention relates to a novel “P. kurroa hydrolyzate extract”, this expression corresponding, in the text hereinbelow, to an extract, by means of an organic solvent, of a root and/or rhizome hydrolyzate of the Picrorhiza kurroa plant, enriched in monophenolic compounds derived from the acid hydrolysis of glycosides present in the plant. These monophenolic compounds are essentially aglycones of Picrorhiza kurroa root and rhizome glycosides.

A subject of the invention is also the use of such an extract in cosmetics, in a topical formulation, to obviate the visible signs of skin aging. This use of Picrorhiza kurroa, and in particular of the extract resulting from the process of extraction of this plant according to the invention for improving the condition of the skin at the first signs of aging of the skin, constitutes an innovation relative to the uses known in the prior art.

A subject of the invention is also the use of such an extract in a topical formulation for counteracting the manifestations of pathological skin aging described above. This use of Picrorhiza kurroa, and in particular of the extract resulting from the process of extraction of this plant according to the invention, for improving the condition of the skin during the early stages of dermatoporosis, constitutes an innovation relative to the uses known in the prior art.

According to the invention, an extract is defined by points 1 and 12 below.
1. An extract of a plant of the genus Picrorhiza, chosen from Picrorhiza kurroa, Picrorhiza scrophulariiflora and Neopicrorhiza scrophulariiflora, obtained by extraction with a first organic solvent of an acid aqueous hydrolyzate of elements from the underground part of the plant, said elements being chosen from rhizomes and roots.
12. The extract as defined in one of points 1 to 4 or the mixture as defined in point 5, for use as a medicament, notably for use in the treatment and prevention of dermatoporosis intrinsic or secondary to the use of corticosteroids and/or for use in a treatment by local administration.

Different extract embodiments are defined by points 2 to 4 below.
2. The extract as defined in point 1, characterized in that said plant is Picrorhiza kurroa.
3. The extract as defined in point 1 or 2, characterized in that the hydrolysis step is performed in an aqueous medium comprising 10% to 30% by volume of a second organic solvent that is totally miscible with water and 1 to 5 M/l of HCl.
4. The extract as defined in one of the preceding points, characterized in that the first solvent is ethyl acetate.

According to the invention, a mixture is defined by point 5 below.
5. A mixture comprising several extracts as defined in one of the preceding points.

According to the invention, a composition is defined by points 6 and 12 below.
6. A composition, characterized in that it comprises:
   an extract as defined in one of points 1 to 4, or
   a mixture as defined in point 5,
   in a dermatologically acceptable vehicle.
12. The extract as defined in any one of points 6 to 10, for use as a medicament, notably for use in the treatment and prevention of dermatoporosis intrinsic or secondary to the use of corticosteroids and/or for use in a treatment by local administration.

Different composition embodiments are defined by points 7 to 10 below.
7. The composition as defined in the preceding point, in which the extract is an extract of Picrorhiza kurroa at a concentration of between 0.05% and 1% (by mass).
8. The composition as defined in point 6 or 7, comprising one or more aglycones of the extract or extract mixture, in particular vanillic acid (for example between 0.1% and 0.5% by mass) and/or ferulic acid (for example between 0.1% and 0.5% by mass) and/or caffeic acid (for example between 0.1% and 0.5% by mass) and/or coumaric acid (for example between 0.1% and 0.5% by mass) and/or apocynin (for example between 0.5% and 1% by mass).
9. The composition as defined in one of points 6 to 8, also containing an AhR receptor agonist ligand or proligand chosen from L-tryptophan and tryptophan derivatives metabolizable by phase I enzymes.
10. The composition as defined in one of points 6 to 9, characterized in that the composition is intended for topical cutaneous application.

According to the invention, a use is defined by point 11 below.
11. The cosmetic use of a composition as defined in one of the preceding points 6 to 10, in topical formulation, for treating skin aging.

According to the invention, a process for producing an extract is defined by point 13 below.
13. A process for producing an extract of a plant of the genus Picrorhiza, chosen from Picrorhiza kurroa, Picrorhiza scrophulariiflora and Neopicrorhiza scrophulariiflora, as defined in one of points 1 to 4 or 12, or a mixture of extracts as defined in point 5, the process comprising the following steps:
   grinding the plant roots and/or rhizomes to obtain a powder,
   placing the powder in an aqueous solution of an acid,
   performing an extraction of the mixture using a solvent which is immiscible or only sparingly miscible with water,
   evaporating off the solvent.

Other features of the invention will be seen by a person skilled in the art in the description below of a mode of implementation and from the accompanying figures.

### BRIEF DESCRIPTION OF THE DRAWINGS

A mode of implementation of the invention is described below with reference to the figures:

FIG. 1A is a chromatogram of a Picrorhiza kurroa extract obtained without acid hydrolysis,

FIG. 1B is a chromatogram of a Picrorhiza kurroa extract obtained following acid hydrolysis, according to the invention,

FIG. 2 shows filopodia formation in four differently treated in vitro cultures.

### DETAILED DESCRIPTION OF PARTICULAR EMBODIMENTS

The chemical species sought in the context of the invention are notably aglycones of glycosides produced by the plant. Their use in a topical formulation involves including them in the form of free aglycones, as the skin does not hydrolyze the glycosides, and furthermore, as they are polar, they do not penetrate the skin.

The raw material used is a powder obtained by grinding the roots and rhizome of the Picrorhiza kurroa plant, or one of the Picrorhiza scrophulariiflora or Neopicrorhiza scrophulariiflora plants, or of a mixture thereof.

This powder is suspended in an aqueous solution of an acid to perform hydrolysis of the glycosides present in the powder. Dilute hydrochloric acid, at a concentration of about 1-5 mol/L, typically 2-3 mol/L, is suitable for performing this hydrolysis step. Other acids may be used, but hydrochloric acid is preferred as its conjugate base, the Cl-ion, is chemically neutral with respect to possible chemical reactions with other constituents of the treated mixture, compared with the conjugate bases of certain other acids such as sulfate or acetate. According to one mode of implementation, a minor proportion, of about 10-30% by weight, and preferably 20-25% by weight, of a totally water-miscible organic solvent, such as ethanol or methanol, is added to the hydrolysis medium to improve the yield. According to one mode of implementation, the hydrolysis is performed at a temperature above room temperature, chosen between 60° C. and 95° C. After the hydrolysis step, which usually lasts between 30 minutes and 3 hours, the reaction mixture can be cooled.

The reaction mixture is then extracted one or more times with a water-immiscible or sparingly water-miscible solvent. Preferably, a slightly polar organic solvent is used (typically 3 times the volume of the aqueous phase). According to one mode of implementation, the extraction solvent is ethyl acetate. The solvent is then evaporated off under reduced pressure, thus obtaining the hydrolyzate extract according to the invention.

The topical formulation, which is the subject of the invention, must contain the hydrolyzate extract according to the invention in a dermatologically acceptable vehicle. This vehicle may be chosen from ointments, pomades, creams, milks and lotions known to those skilled in the art. Preferably, the formulation comprises between 0.05% and 1% (by mass) of P. kurroa hydrolyzate extract in a presentation form favoring the penetration of polar organic compounds.

In one implementation variant, the topical formulation forming the subject of the invention also contains an AhR receptor agonist ligand or proligand chosen from L-tryptophan and tryptophan derivatives metabolizable by phase I enzymes.

In one implementation variant of the topical formulation, one or more aglycones of the extract forming the subject of the invention are added in pure form.

### EXAMPLES

### Example 1: Preparation of an Extract of Picrorhiza kurroa Hydrolyzate According to the Invention

The starting point is a commercially available powder, Kutki from HerbsForever, for example, obtained by grinding the roots and rhizome of the Picrorhiza kurroa plant.

This powder is partially dissolved in a mixture comprising 25% methanol and 75% hydrochloric acid at 2.7 mol/L, with a volume of 10 ml per g of powder. The whole is heated for one hour at 80° C., allowing hydrolysis of the glycosides. After returning to room temperature, the mixture is extracted twice with 3 volumes of ethyl acetate. The solvent is evaporated off under reduced pressure, thus obtaining the extract enriched in monophenolic compounds.

### Comparative Example 2: Preparation of an Extract of Picrorhiza kurroa without Acid Hydrolysis

The starting point is the same powder, obtained by grinding the roots and rhizome of the Picrorhiza kurroa plant, as in Example 1.

Extraction is performed using a polar organic solvent, i.e. methanol or ethanol. The powder is partially dissolved in a mixture comprising 10 ml of methanol per gram of powder. The whole is heated for one hour at 40° C., allowing extraction of polar organic compounds.

Chromatographic Analysis of the Two Extracts of Picrorhiza kurroa

The two Picrorhiza kurroa extracts are analyzed by HPLC-UV (Agilent) on a C18 Nucleodur® Pyramid 3 μm column (Macherey-Nagel) using a linear gradient of mobile phases at constant flow (Table 1):

TABLE 1
Mobile phase concentration gradient for HPLC analysis
of P. kurroa extracts. A: 0.2% acetic acid (aqueous);
B: acetonitrile; C: methanol. Detection is performed
by simultaneous measurement of absorbance at the following
wavelengths: 260 nm, 275 nm, 310 nm, 322 nm.

| | | | | Flow rate |
|---|---|---|---|---|
| Time (min) | % A | % B | % C | (ml/ min) |
| 0 | 50 | 50 | 0 | 0.800 |
| 9 | 50 | 50 | 0 | 0.800 |
| 12 | 0 | 100 | 0 | 0.800 |
| 20 | 0 | 100 | 0 | 0.800 |
| 22 | 0 | 0 | 100 | 0.800 |
| 35 | 0 | 0 | 100 | 0.800 |

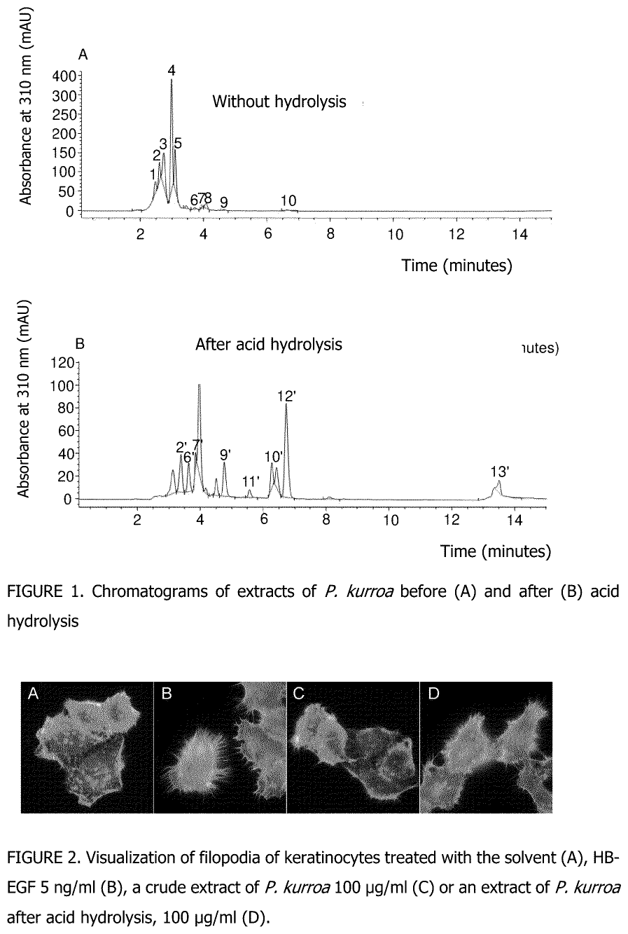

Chromatograms of the two Picrorhiza kurroa extracts without and after acid hydrolysis show (FIG. 1):
in FIG. 1A, without hydrolysis: large early peaks are seen (3, 4, 5), the absorption spectra of which match those of later peaks that are seen in the chromatogram of the extract after acid hydrolysis (FIG. 1B). These include, inter alia, caffeic, vanillic, coumaric and ferulic acids, and also certain derivatives thereof;
in FIG. 1B (after hydrolysis), peaks 3, 4, 5 are replaced by new, later peaks with identical absorption spectra: this is due to the hydrolysis of glycosides which have released aglycones, responsible for the absorption spectra obtained, since the glycosidic (sugar) parts of glycosides do not absorb light beyond 220 nm; aglycones being less polar than glycosides, they are retained more by the separation column and reach the detector later.

Peaks 3, 4, 5 (A) emerge earlier—and are thus more polar (glycosides)—and have the same absorption spectra as peaks 6-6′, 8-8′, 10-10′-13′, respectively (A, B). These are probably glycosides whose aglycones are seen mainly after acid hydrolysis. Peak identification: 2, 2′: caffeic acid; 6, 6′: vanillic acid; 7, 7′: coumaric acid; 8, 8′: ferulic acid; 9, 9′: apocynin, 10, 10′: cinnamic acid. Peak 11′ has the same absorption spectrum as vanillic acid (6, 6′), peak 12′ has the same spectrum as ferulic acid (8, 8′), and peak 13′ has the same spectrum as peaks 5, 10 and 10′.

### Example 3: In Vitro Biological Properties of the Extract of P. kurroa Hydrolyzate

Table 2 indicates the intensity of filopodia formation (number and surface area) in vitro in four keratinocyte cultures under four operating conditions, after treatment with:
a solvent,
HB-EGF (heparin-binding epidermal growth factor),
Picrorhiza kurroa extract obtained without hydrolysis, and
Picrorhiza kurroa extract obtained following hydrolysis.

HB-EGF (positive control) has an intense effect, the hydrolyzed Picrorhiza kurroa extract has a medium effect, and the solvent and non-hydrolyzed extract have no effect.

The test is performed using a human keratinocyte culture divided into four batches. Cells are treated for 1 h with the products to be tested, fixed in 4% formalin, and then stained with rhodamine-conjugated α-phalloidin (α-phalloidin has high affinity for actin filaments, and its conjugation to rhodamine makes actin filaments fluorescent [540 nm; 565 nm]). The cell morphology is then analyzed under a fluorescence microscope (FIG. 2).
A, solvent, negative (no stimulation);
B, HB-EGF (heparin-binding epidermal growth factor), reference epidermal trophic factor, 5 ng/ml;
C, Picrorhiza kurroa extract obtained without hydrolysis, 100 μg/ml;
D, Picrorhiza kurroa extract obtained following hydrolysis, 100 μg/ml. Table 2 shows filopodia production.

TABLE 2
Visual evaluation of the production of filopodia (scale from 0 to 3)

| | | Crude extract of P. kurroa | Extract of P. kurroa |
|---|---|---|---|
| | HB-EGF | obtained without | obtained following |
| Solvent | (5 ng/ml) | hydrolysis (100 μg/ml) | hydrolysis (100 μg/ml) |
| 0 | 3 | 0-1 | 1-2 |

### Example 4: Clinical Study on Subjects Presenting with Embrittled Skin: Counting of Senile Purpuras

The first clinical signs of this fragility are most often superficial hematomas which appear spontaneous, but are in fact due to minor everyday traumas which until then had been tolerated. These are known as senile purpuras. They constitute a good means of testing the protective effect of a topical preparation when they reduce in number and frequency during prolonged application of said topical preparation.

To this end, a composition intended for topical cutaneous application (see Table 3), according to Example 4, was applied to eleven dermatoporosis patients on the posterior face of the forearms—the preferred site for senile purpuras—for several weeks (Table 4). The preparation was applied every evening for at least 12 weeks. The number of senile purpuras was recorded weekly (and the area photographed by the patient on his cell phone). Table 4 shows that, prior to the start of topical application, the average number of senile purpuras present at the 22 sites analyzed was 6.8. These mean numbers reduced each month to 4.2 after one month, then to 2.2 after two months, and then to 1.6 after three months. These observations indicate a noteworthy effect of the preparation.

TABLE 3
Composition of the topical formulation according to Example 4.

Water
Soybean oil
Glycerol
Span ® 60
Myrj 52
Caprylic/capric triglycerides
Petroleum jelly
Cetyl alcohol
Isopropyl myristate
Glyceryl monooleate
Extract of P. kurroa obtained following hydrolysis
α-Tocopherol
Ethanol
Methyl paraben
Sodium hyaluronate
Xanthan gum
All-trans retinal

TABLE 4
Senile purpuras

Number of senile purpuras

| | | After 1 | After 2 | After 3 |
|---|---|---|---|---|
| Site analyzed | Before | month | months | months |
| Left forearm | 4 | 3 | 2 | 1 |
| Right forearm | 5 | 3 | 1 | 1 |
| Left forearm | 4 | 3 | 2 | 1 |
| Right forearm | 3 | 1 | 1 | 1 |
| Left forearm | 10 | 6 | 4 | 3 |
| Right forearm | 9 | 7 | 5 | 2 |
| Left forearm | 4 | 3 | 2 | 1 |
| Right forearm | 3 | 2 | 2 | 2 |
| Left forearm | 5 | 2 | 2 | 2 |
| Right forearm | 3 | 3 | 1 | 1 |
| Left forearm | 9 | 7 | 4 | 2 |
| Right forearm | 10 | 6 | 3 | 3 |
| Left forearm | 20 | 10 | 3 | 3 |
| Right forearm | 11 | 7 | 2 | 4 |
| Left forearm | 4 | 2 | 1 | 1 |
| Right forearm | 3 | 1 | 1 | 1 |
| Left forearm | 8 | 3 | 2 | 1 |
| Right forearm | 12 | 6 | 3 | 1 |
| Left forearm | 3 | 2 | 1 | 1 |
| Right forearm | 2 | 2 | 1 | 1 |
| Left forearm | 8 | 5 | 2 | 1 |
| Right forearm | 9 | 8 | 3 | 2 |
| Total number of | 149 | 92 | 48 | 36 |
| purpuras | | | | |
| Number of sites | 22 | 22 | 22 | 22 |
| tested | | | | |
| Mean number of | 6.8 | 4.2 | 2.2 | 1.6 |
| purpuras | | | | |

### Example 5: Clinical Study on Subjects Presenting with Embrittled Skin: Analysis of the Cutaneous Thickness by Ultrasonography

Dermatoporosis is characterized by a loss of extracellular and cellular matrix. Thus, just as osteodensimetry is used to diagnose osteoporosis, it is possible to evaluate dermatoporotic skin by ultrasonography, for example using the 1-200 Epi Scan SRL ultrasonography instrument. This examination allows objectification of the loss of cutaneous extracellular and cellular matrix, resulting in a reduction in the thickness of a defined dermoepidermal area. Thus, the thickness measured on the dorsal faces of the forearms is 1.5 mm in the normal state, but may be reduced to less than 0.5 mm, representing a loss of 67% in dermatoporotic skin. It has been reported that effective topical treatments with retinaldehyde and hyaluronic acid fragments could induce a gain of 20% to 30% in dermatoporosis patients (Kaya et al., 2018).

To this end, a composition intended for topical cutaneous application according to the invention was applied by eight subjects suffering from dermatoporosis (intrinsic in four subjects, and after corticosteroid use in four others), to the posterior face of the forearms, the anterior face of the legs and the neckline—conventional sites of dermatoporosis—for several months (up to six months, Table 5). The preparation was applied every evening for at least 8 weeks. Measurements were performed using an Episcan I-200 ultrasonic scanner. Table 5 shows that the average increase in skin thickness was 14.7% at two months, 19.3% at three months, 38.6% at four months, 18.6% at five months and 30.1% at six months. These results are comparable to those obtained with effective topical treatments based on retinal and hyaluronic acid fragments, and indicate the activity of the preparation according to the invention.

TABLE 5
Evaluation of the epidermal thickness

| | | Percentage increase as a | | | | |
|---|---|---|---|---|---|---|
| | | function of the application time | | | | |
| Patient | Location | 2 months | 3 months | 4 months | 5 months | 6 months |
| 1 | RFA | 13 | | | 15 | |
| | LFA | 8 | | | 21 | |
| | RL | 7 | | | 21 | |
| | LL | 7 | | | 17 | |
| | NL | 3 | | | 10 | |
| 2 | RFA | | 27 | | | 53 |
| | LFA | | 26 | | | 51 |
| | RL | | 15 | | | 27 |
| | LL | | 15 | | | 38 |
| | NL | | 13 | | | 39 |
| 3 | RFA | | −17 | | | −7 |
| | LFA | | 32 | | | 36 |
| 4 | RFA | | 31 | | | |
| | LFA | | 32 | | | |
| 5 | RFA | 2 | | | | |
| | LFA | 1 | | | | |
| | RL | 6 | | | | |
| | LL | −2 | | | | |
| | NL | 9 | | | | |
| 6 | RFA | 37 | | 45 | | |
| | LFA | 36 | | 41 | | |
| | RL | 33 | | 42 | | |
| | LL | 30 | | 35 | | |
| | NL | 24 | | 30 | | |
| 7 | RFA | 22 | | | | 28 |
| | LFA | 5 | | | | 14 |
| | RL | 29 | | | | 32 |
| | LL | 10 | | | | 20 |
| 8 | RFA | 13 | | | 22 | |
| | LFA | 16 | | | 24 | |

| | | | | | |
|---|---|---|---|---|---|
| Total | 309 | 174 | 193 | 130 | 331 |
| Number of | 21 | 9 | 5 | 7 | 11 |
| sites tested | | | | | |
| Mean increase | 14.7 | 19.3 | 38.6 | 18.6 | 30.1 |

RFA: right forearm, LFA: left forearm, RL: right leg, LL: left leg, NL: neckline.

### REFERENCES

[1] Saurat J H. Quand la peau devient insuffisante. Méd Hyg. 2004; 476.
[2] Kaya G, Kaya A, Sorg O, Saurat J H. Dermatoporosis: a further step to recognition. J Eur Acad Dermatol Venereol. 2018; 32; 189-191.
[3] Humbert P, Fanian F, Lihoreau T, Jeudy A, Pierard G E. Bateman purpura (dermatoporosis): a localized scurvy treated by topical vitamin C-double-blind randomized placebo-controlled clinical trial. J Eur Acad Dermatol Venereol. 2018; 32; 323-328.
[4] Kaya G, Saurat J H. Dermatoporosis: a chronic cutaneous insufficiency/fragility syndrome. Clinicopathological features, mechanisms, prevention and potential treatments. Dermatology. 2007; 215; 284-294.
[5] Kluger N. Dermatoporosis and vitamin C deficiency. J Eur Acad Dermatol Venereol. 2018.
[6] Kluger N, Schaan K, Girard C, Guillot B, Dereure O. Hématomes multiples sur dermatoporose et carence en vitamine C. Nouv Dermatol. 2010; 29; 371-373.
[7] Saurat J H. Dermatoporosis. The functional side of skin aging. Dermatology. 2007; 215; 271-272.
[8] Mengeaud V, Dautezac-Vieu C, Josse G, Vellas B, Schmitt A M. Prevalence of dermatoporosis in elderly French hospital in-patients: a cross-sectional study. Br J Dermatol. 2012; 166; 442-443.
[9] WO 2005/082327 A1
[10] WO 2009/093207
[11] Krupashree K, Kumar K H, Rachitha P, Jayashree G V, Khanum F. Chemical composition, antioxidant and macromolecule damage protective effects of Picrorhiza kurroa Royle ex Benth. South Afr J Bot. 2014; 94; 249-254.

1. An extract of a plant of the genus Picrorhiza,
wherein the plant is selected from the group consisting of Picrorhiza kurroa, Picrorhiza scrophulariiflora and Neopicrorhiza scrophulariiflora,
wherein the extract has been obtained by extraction by hydrolysis with a first organic solvent of an acid aqueous hydrolyzate of elements from the underground part of the plant, wherein the elements from the underground part of the plant are selected from the group consisting of rhizomes and roots.

2. The extract as claimed in claim 1, wherein the plant is Picrorhiza kurroa.

3. The extract as claimed in claim 1, wherein the hydrolysis is performed in an aqueous medium comprising in a range of from 10% to 30% by volume of a second organic solvent that is totally miscible with water and 1 to 5 M/l of HCl.

4. The extract as claimed in claim 1, wherein the first solvent is ethyl acetate.

5. A mixture comprising several extracts as claimed in claim 1.

6. A composition comprising:
an extract as claimed in claim 1, and
a dermatologically acceptable vehicle.

7. The composition as claimed in claim 6, wherein the extract is an extract of Picrorhiza kurroa at a concentration in a range of from 0.05% to 1% by mass.

8. The composition as claimed in claim 6, comprising one or more aglycones of the extract.

9. The composition as claimed in claim 6, also containing an AhR receptor agonist ligand or proligand selected from the group consisting of L-tryptophan and tryptophan derivatives metabolizable by phase I enzymes.

10. The composition as claimed in claim 6, wherein the composition is adapted for topical cutaneous application.

11. A method of treating skin aging, comprising applying to the skin a topical formulation comprising an effective amount of a composition as claimed in claim 6.

12. A method of treating or preventing dermatoporosis intrinsic or secondary to use of corticosteroids, comprising administering a composition as claimed in claim 6.

13. A process for producing an extract of a plant of the genus Picrorhiza, wherein the plant is selected from the group consisting of Picrorhiza kurroa, Picrorhiza scrophulariiflora and Neopicrorhiza scrophulariiflora, wherein the process comprises:
grinding plant roots and/or rhizomes to obtain a powder,
placing the powder in an aqueous solution of an acid,
performing an extraction of the mixture using a solvent which is immiscible or only sparingly miscible with water,
evaporating off the solvent,
so as to obtain the extract as claimed in claim 1.

14. The composition as claimed in claim 8, comprising vanillic acid in a range of from 0.1% and 0.5% by mass and/or ferulic acid in a range of from 0.1% and 0.5% by mass and/or caffeic acid in a range of from 0.1% and 0.5% by mass and/or coumaric acid in a range of from 0.1% and 0.5% by mass and/or apocynin in a range of from 0.5% and 1% by mass.

15. A composition comprising:
a mixture as claimed in claim 6, and
a dermatologically acceptable vehicle.

16. The composition as claimed in claim 15, comprising one or more aglycones of at least one of the extracts.

17. The composition as claimed in claim 15, also containing an AhR receptor agonist ligand or proligand selected from the group consisting of L-tryptophan and tryptophan derivatives metabolizable by phase I enzymes.

18. The composition as claimed in claim 15, wherein the composition is adapted for topical cutaneous application.

19. A method of treating skin aging, comprising applying to the skin a topical formulation comprising an effective amount of a composition as claimed in claim 15.

20. A method of treating or preventing dermatoporosis intrinsic or secondary to use of corticosteroids, comprising administering a composition as claimed in claim 15.
